(19) United States
(12) Patent Application Publication
(10) Pub. No.: US 2020/0330399 A1
(43) Pub. Date: Oct. 22, 2020

(54) COMBINATION THERAPY COMPRISING A POLYUNSATURATED KETONE AND A CORTICOSTEROID

(71) Applicant: AVEXXIN AS, Trondheim (NO)

(72) Inventors: Berit JOHANSEN, Trondheim (NO); Astrid Jullumstrø FEUERHERM, Trondheim (NO)

(73) Assignee: AveXxin AS, Trondheim (NO)

(21) Appl. No.: 16/306,126

(22) PCT Filed: Jun. 5, 2017
(86) PCT No.: PCT/EP2017/063629

§ 371 Date: Nov. 30, 2018

(30) Foreign Application Priority Data
Jun. 3, 2016 (GB) 1609719.8
Jul. 29, 2016 (GB) 1613179.9
Mar. 17, 2017 (GB) 1704281.3

(51) Int. Cl. A61K 31/121; A61K 31/573; A61K 31/4164; A61K 31/702; A61K 31/60; A61K 31/047; A61K 9/06

(52) U.S. Cl. CPC A61K 31/121; A61K 31/573; A61K 31/4164; A61K 9/06; A61K 31/60; A61K 31/047; A61K 31/702

(57) ABSTRACT

A synergistic pharmaceutical composition for simultaneous, parallel, sequential or separate use comprising a polyunsaturated ketone, a corticosteroid and, optionally, a secosteroid partner calciptriol. The composition has utility in the treatment and prevention of skin disorders.

This invention relates to a pharmaceutical composition comprising certain polyunsaturated long-chain ketones in combination with certain corticosteroids such as betamethasone or a pharmaceutically acceptable salt, or a hydrate or solvate thereof. The invention also relates to the use of said pharmaceutical composition for the treatment or prevention of skin conditions such as dermatitis and psoriasis.

### BACKGROUND

This invention is concerned with a combination therapy for the treatment of certain skin conditions such as psoriasis and dermatitis. In its broadest sense, dermatitis is inflammation of the skin. It is a common and disfiguring skin condition which requires quick and efficient treatment. Dermatitis symptoms vary, however, with the different forms of the condition. Symptoms vary from skin rashes to bumpy rashes through to flaky skin and blisters. Although different types of dermatitis have varying symptoms, there are certain signs that are common for all of them, including redness of the skin, swelling, itching, skin lesions and sometimes oozing and scarring.

Also, the area of the skin on which the symptoms appear tends to be different with every type of dermatitis. Types of dermatitis are classified according to the cause of the condition. Contact dermatitis is caused by an allergen or an irritating substance. Irritant contact dermatitis accounts for 80% of all cases of contact dermatitis.

Atopic dermatitis is very common worldwide and increasing in prevalence. Atopic dermatitis is a type of eczema and is an inflammatory, chronically relapsing, non-contagious and itchy skin disorder.

Other less common forms of dermatitis include dermatitis herpetiformis. It is characterized by intensely itchy, chronic papulovesicular eruptions, usually distributed symmetrically on extensor surfaces such as the back of neck, scalp, elbows, knees, back, hairline, groin or face.

Seborrheic dermatitis is a dermatitis that occurs in the vicinity of sebaceous glands and is caused by sebum over production. The condition tends to give a scaly, flaky skin condition.

Stasis dermatitis is an inflammation on the lower legs which is caused by build-up of blood and fluid and it is more likely to occur in people with varicose veins.

Other common skin disorders include psoriasis. This is an autoimmune induced, chronic disease of skin characterised by red, itchy and scaly skin patches. Skin disorders in general and dermatitis and psoriasis in particular are disfiguring and can lead to reluctance of a sufferer to let people see their condition. Successful treatments of these skin disorders are therefore sought.

A common treatment for skin disorders is administration of one or more topical corticosteroids. The present inventors have now found that the combination of certain polyunsaturated ketones and certain corticosteroids such as betamethasone or a pharmaceutically acceptable salt, or a hydrate or solvate results in a synergistic improvement in performance.

### SUMMARY OF INVENTION

Thus, viewed from one aspect the invention provides a pharmaceutical composition comprising:

(A) at least one compound of formula (I):

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group;

or a pharmaceutically acceptable salt, or a hydrate or solvate thereof; and

(B) one or more corticosteroid partners, preferably selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone and prednicarbate or a pharmaceutically acceptable salt, or a hydrate or solvate thereof.

In a preferred embodiment, betamethasone or a pharmaceutically acceptable salt, or a hydrate or solvate thereof is the corticosteroid partner.

Viewed from another aspect the invention provides a pharmaceutical kit composition for simultaneous, in parallel, sequential or separate use comprising a first composition comprising at least one compound (I) as herein defined and a pharmaceutically-acceptable diluent or carrier, and a second composition comprising at least one compound (B) as the corticosteroid partner as herein defined such as betamethasone or a pharmaceutically acceptable salt, or a hydrate or solvate thereof and a pharmaceutically-acceptable diluent or carrier.

In particular, the invention relates to a pharmaceutical composition, or kit as herein before defined in which the compound of formula (I) is:

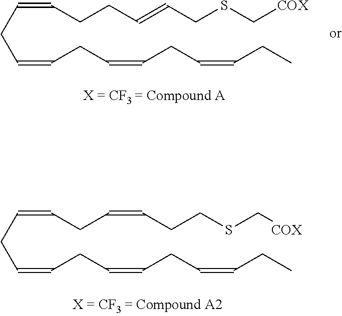

or a pharmaceutically acceptable salt, or a hydrate or solvate thereof. In particular, the corticosteroid partner (B) is betamethasone or a pharmaceutically acceptable salt, or a hydrate or solvate thereof.

At least one other corticosteroid partner may be combined with the betamethasone to achieve intended results, for example, 1 or 2 of such compounds. Alternatively, the betamethasone (including a salt, hydrate or solvate thereof) may be substituted by at least one other corticosteroid partner, for example, 1 or 2 of such other compounds (including pharmaceutically acceptable salts, or hydrates or solvates of such compounds).

Viewed from another aspect the invention provides a pharmaceutical composition as hereinbefore defined for use in the treatment or prevention of a skin disorder such as psoriasis or dermatitis.

Viewed from another aspect the invention provides a method of treating or preventing a skin disorder such as psoriasis or dermatitis in an animal subject, for example, a mammal such as rodent (mouse, rat, rabbit), monkey (or other non-human primate), pig or other laboratory animal used as a model to study skin disorders. Another suitable mammalian subject is a patient in need thereof. In one embodiment, the invention comprises administering to said subject (e.g. a human patient) an effective amount of a pharmaceutical composition as herein before defined.

Viewed from another aspect the invention provides a method of treating, such as reducing symptoms of, or preventing a skin disorder such as psoriasis or dermatitis, in a patient in need thereof comprising administering to said patient, preferably a human, an effective amount of at least one compound of formula (I) and simultaneously, in parallel, separately or sequentially administering to said patient an effective amount of at least one compound (B) (e.g., 1, 2 or 3 of such compounds) as herein defined. In sequential administration either compound can be administered first.

Viewed from another aspect the invention provides a method of treating, such as reducing symptoms of, or preventing a skin disorder such as psoriasis or dermatitis, in a patient in need thereof comprising:
(i) identifying a patient who has received either a compound of formula (I) or a compound (B);
(ii) administering to said patient an effective amount of either at least one of a compound (B) as herein defined or at least one of a compound of formula (I) as herein before defined so that said patient is administered with both at least one compound of formula (I) and at least one compound (B).

In preferred embodiments, 1, 2 or 3 of compound B will be suitable for use with the invention with 1 or 2 of compound B being preferred for many invention applications.

Viewed from another aspect the invention provides use of a pharmaceutical composition as hereinbefore defined in the manufacture of a medicament for treating or preventing a skin disorder such as psoriasis or dermatitis.

Viewed from another aspect the invention provides a process for the preparation of a pharmaceutical composition as hereinbefore defined comprising blending at least one compound of formula (I) or a pharmaceutically acceptable salt, or a hydrate or solvate thereof and at least one compound (B) or a pharmaceutically acceptable salt, or a hydrate or solvate thereof in the presence of at least one pharmaceutical excipient.

### Definitions

The term lower alkyl is used herein to refer to C1-6 alkyl groups, preferably C1-4 alkyl groups, especially C1-3 alkyl groups. These alkyl groups can be linear or branched, preferably linear.

In one embodiment, the invention relates to a pharmaceutical composition in which at least one compound (I) and at least one corticosteroid partner (e.g., 1, 2, or 3 of such compounds) are blended together in a single composition. The invention also relates to a pharmaceutical composition in the form of a kit in which the active compounds are provided in separate compositions but are designed for administration simultaneously, in parallel), separately or sequentially. Any method for treating or preventing a skin disorder as defined herein encompasses simultaneous, in parallel, separate or sequential administration of the active components or administration of the composition of the invention.

The pharmaceutical composition of the invention is a “combination”, which means either a fixed combination in one dosage unit form, or non fixed combination such as a kit of parts for combined administration where a compound of the formula (I) and at least one corticosteroid partner(s) (e.g., 1, 2 or 3 of such compounds) may be administered independently at the same time (e.g., in parallel) or separately within time intervals, especially where these time intervals allow that the combination partners show a cooperative and preferably a synergistic effect.

Thus a “pharmaceutical composition” as used herein means a product suitable for pharmaceutical use that results from the mixing, admixing or combining more than one active ingredient and includes both fixed and non-fixed combinations of the active ingredients. The term “fixed combination” or “fixed dose” means that the active ingredients, e.g. a compound of formula (I) and a corticosteroid partner such as betamethasone, are both administered to a patient simultaneously in the form of a single entity or dosage. The pharmaceutical composition can also be a “non-fixed combination” which means that the active ingredients, e.g. a compound of formula (I) and the combination partner betamethasone are both administered to a patient as separate entities either simultaneously, in parallel, concurrently or sequentially with no specific time limits, wherein such administration provides therapeutically effective levels of the two compounds in the body of the animal in need thereof.

A “corticosteroid partner” as used herein means a synthetic or semisynthetic corticosteroid generally suitable for intended goals of the invention. Preferred corticosteroid partners include the following: betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a pharmaceutically acceptable salt, or a hydrate or solvate thereof. Betamethasone and its pharmaceutically acceptable salts, hydrates and solvates thereof are especially preferred corticosteroid partners.

All discussion below relating to preferred compounds of the invention is equally applicable to both these aspects of the invention.

### DETAILED DESCRIPTION

This invention concerns a combination therapy of a least one compound of formula (I) and at least one corticosteroid partner, in particular 1, 2 or 3 of such compounds with 1 or 2 compounds being preferred for many invention applications. In a preferred embodiment, betamethasone or a pharmaceutically acceptable salt, or a hydrate or solvate thereof is the corticosteroid partner. We have surprisingly found that this combination therapy results in synergy. Our results demonstrate a reduction in the proliferation and viability of HaCaT cells, the pharmaceutical composition offering a larger decrease than could have been expected from the use of compounds individually, i.e. the combination of the compounds produces an overall effect that is greater than the sum of the individual elements.

### Pharmaceutical Composition of the Invention

The invention relies on the therapeutic combination of at least one compound of formula (I) and at least one corticosteroid partner such as betamethasone or a pharmaceutically acceptable salt, or a hydrate or solvate thereof. The compound of formula (I) is

R-L-CO—X (I)

wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group; or a salt, or a hydrate or solvate thereof.

The group R preferably comprises 5 to 9 double bonds, preferably 5 or 8 double bonds, e.g. 5 to 7 double bonds such as 5 or 6 double bonds. These bonds should be non-conjugated. It is also preferred if the double bonds do not conjugate with the carbonyl functionality.

The double bonds present in the group R may be in the cis or trans configuration however, it is preferred if the majority of the double bonds present (i.e. at least 50%) are in the cis configuration. In further advantageous embodiments all the double bonds in the group Rare in the cis configuration or all double bonds are in the cis configuration except the double bond nearest the carbonyl group which may be in the trans configuration.

The group R may have between 10 and 24 carbon atoms, preferably 12 to 20 carbon atoms, especially 17 to 19 carbon atoms.

Whilst the R group can be interrupted by at least one heteroatom or group of heteroatoms, this is not preferred and the R group backbone preferably contains only carbon atoms.

The R group may carry up to three substituents, e.g. selected from halo, C₁₋₆ alkyl e.g. methyl, or C₁₋₆ alkoxy. If present, the substituents are preferably non-polar, and small, e.g. a methyl group. It is preferred however, if the R group remains unsubstituted.

The R group is preferably an alkylene group.

The R group is preferably linear. It preferably derives from a natural source such as a long chain fatty acid or ester. In particular, the R group may derive from AA, EPA or DHA.

Thus, viewed from another aspect the invention employs a compound of formula (I′)

R-L-CO—X (I′)

wherein R is a C₁₀₋₂₄ unsubstituted unsaturated alkylene group said group comprising at least 4 non-conjugated double bonds;

L is a linking group forming a bridge of 1 to 5 atoms between the R group and the carbonyl CO wherein L comprises at least one heteroatom in the backbone of the linking group; and

X is an electron withdrawing group or a salt thereof.

Ideally R is linear. R is therefore preferably an unsaturated C₁₀₋₂₄ polyalkylene chain.

The linking group L provides a bridging group of 1 to 5 backbone atoms, preferably 2 to 4 backbone atoms between the R group and the carbonyl, such as 2 atoms. The atoms in the backbone of the linker may be carbon and/or be heteroatoms such as N, O, S, SO, SO₂. The atoms should not form part of a ring and the backbone atoms of the linking group can be substituted with side chains, e.g. with groups such as C₁₋₆ alkyl, oxo, alkoxy, or halo.

Preferred components of the linking group are —CH₂—, —CH(C₁₋₆alkyl)-, —N(C₁₋₆alkyl)-, —NH—, —S—, —O—, —CH═CH—, —CO—, —SO—, —SO₂— which can be combined with each other in any (chemically meaningful) order to form the linking group. Thus, by using two methylene groups and an —S— group the linker —SCH₂CH₂— is formed. It will be appreciated that at least one component of the linker provides a heteroatom in the backbone.

The linking group L contains at least one heteroatom in the backbone. It is also preferred if the first backbone atom of the linking group attached to the R group is a heteroatom or group of heteroatoms.

It is highly preferred if the linking group L contains at least one —CH₂— link in the backbone. Ideally the atoms of the linking group adjacent the carbonyl are —CH₂—.

It is preferred that the group R or the group L (depending on the size of the L group) provides a heteroatom or group of heteroatoms positioned α, β, γ, or δ to the carbonyl, preferably β or γ to the carbonyl. Preferably the heteroatom is O, Nor S or a sulphur derivative such as SO.

Highly preferred linking groups L therefore are —NH₂CH₂, —NH(Me)CH₂—, —SCH₂—, —SOCH₂—, or —COCH₂—

The linking group should not comprise a ring.

Highly preferred linking groups L are SCH₂, NHCH₂, and N(Me)CH₂.

Viewed from another aspect the invention employs a compound of formula (II)

R-L-CO—X (II)

wherein R is a linear C₁₀₋₂₄ unsubstituted unsaturated alkylene group said group comprising at least 4 non-conjugated double bonds;

L is —SCH₂—, —OCH₂—, —SOCH₂, or —SO₂CH₂—; and

X is an electron withdrawing group or a salt thereof.

The group X is an electron withdrawing group. Suitable groups in this regard include O—C₁₋₆ alkyl, CN, OCO₂—C₁₋₆ alkyl, phenyl, CHal₃, CHal₂H, CHalH₂ wherein Hal represents a halogen, e. g. fluorine, chlorine, bromine or iodine, preferably fluorine.

In a preferred embodiment the electron withdrawing group is CHal₃, especially CF₃.

Thus, preferred compounds of formula (I) are those of formula (III)

R—Y1-Y2-CO—X (III)

wherein R and X are as hereinbefore defined;

Y1 is selected from O, S, NH, N(C₁₋₆-alkyl), SO or SO₂ and

Y2 is (CH₂) or CH(C₁₋₆ alkyl); or

where n is 1 to 3, preferably 1.

More, preferred compounds of formula (I) are those of formula (IV)

R—Y1-CH₂—CO—X (IV)

wherein R is a linear C₁₀₋₂₄ unsubstituted unsaturated alkylene group said group comprising at least 4 non-conjugated double bonds;

X is as hereinbefore defined (e.g. CF₃); and

Y1 is selected from O, S, SO or SO₂.

Highly preferred compounds for use in the invention are depicted below.

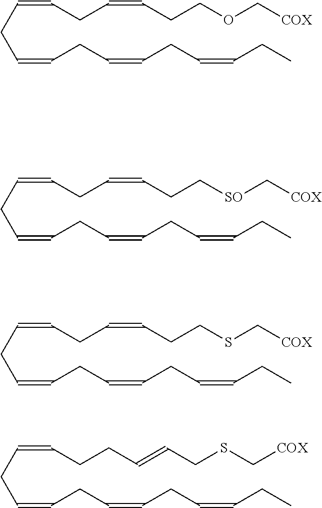

where X is as hereinbefore defined such as CF₃.

The following compounds are highly preferred for use in the invention:

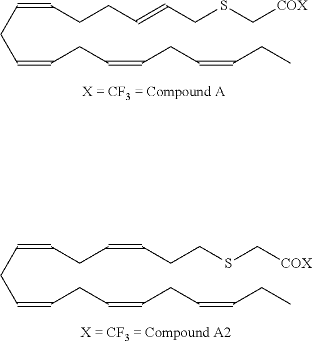

It will be appreciated that the pharmaceutical composition of the invention may comprise one or more than one compound of formula (I) as herein before defined, for example, 1, 2 or 3 of such compounds with 1 or 2 compounds being preferred for most invention applications. Salts, hydrates or solvates of any of these compounds can also be used.

### Corticosteroid

The second component (compound B i.e. the corticosteroid partner) of the composition of the invention is a corticosteroid, preferably a synthetic or semisynthetic corticosteroid, especially betamethasone or a pharmaceutically acceptable salt, or a hydrate or solvate thereof. Betamethasone is a compound of formula:

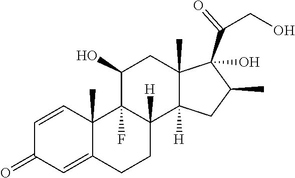

In any composition of the invention the corticosteroid may be present in a salt or non salt form. In particular, in any composition of the invention betamethasone may be present in a salt or non salt form. If a salt form is used, any conventional salt form is possible.

Betamethasone is a known commercial product and any known commercial form of betamethasone can be used. Conveniently, the salt form used is the valerate, acetate or propionate salt. The salt may be a monosalt form, disalt or trisalt form, given the presence of multiple hydroxy groups on which salts can be formed.

If a mono salt form of betamethasone is used, the salt can be present in the 17 position. A disalt form typically comprises a salt at the 17 and 21 positions of the molecule.

Suitable forms include betamethasone diproprionate, betamethasone valerate, betamethasone acetate and betamethasone sodium phosphate.

Whilst the invention is primarily described with reference to betamethasone, it is envisaged that other corticosteroids could also be combined with the compounds of formula (I) to form synergistic combinations.

Possible further corticosteroids include clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone and prednicarbate.

Preferred options include Betamethasone, Clobetasol, Halobetasol, Diflorasone, Fluocinonide, Halcinonide, Amcinonide, Desoximetasone, Triamcinolone, Mometasone, Fluticasone, Halometasone, Hydrocortisone, Flurandrenolide, Desonide, Fluocinolone, and Alclometasone or a salt thereof.

Specific corticosteroid compounds include Clobetasol propionate, Betamethasone dipropionate, Halobetasol propionate, Diflorasone diacetate, Fluocinonide, Halcinonide, Amcinonide, Desoximetasone, Triamcinolone acetonide, Mometasone furoate, Fluticasone propionate, Halometasone, Fluocinolone acetonide, Hydrocortisone valerate, Hydrocortisone butyrate, Flurandrenolide, Triamcinolone acetonide, Mometasone furoate, Fluticasone propionate, Desonide, Fluocinolone acetonide, and Alclometasone dipropionate.

The use of betamethasone type corticosteroids is especially preferred such as betamethasone, dexamethasone and fluocortolone or salts thereof.

In one embodiment, the invention provides a pharmaceutical composition comprising:

(A) a Compound of Formula (I):

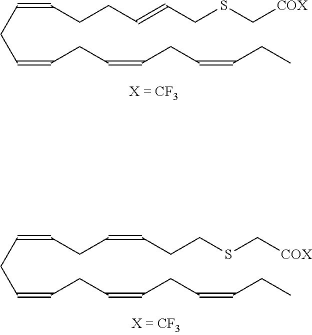

or a salt thereof; and

(B) a corticosteroid partner selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a salt thereof, especially betamethasone, dexamethasone and fluocortolone or a salt thereof.

Alternatively, and as discussed above, the compositions of the invention could comprise betamethasone and additionally comprise one or more further corticosteroids (e.g., 1, 2, or 3) to augment the properties of the composition of the invention. Suitable additional corticosteroids include clobetasol, halometasone, dexamethasone, fluocortolone, desoximetasone, diflorasone, fluocinonide, flurandrenolide, halobetasol, amcinonide, halocinonide, triameinolone, hydrocortisone, aclometasone, fluticasone, mometasone, clocortolone, fluocinolone, desonide, prednisone, prednisolone and prednicarbate or salts thereof. Alternatively, one or more of the aforementioned corticosteroids could be substituted for the betamethasone (including its salts, hydrates and solvates thereof) so long as intended invention results are achieved.

It is also within the scope of the invention to combine the composition of the invention with other compounds conventionally used in conjunction with corticosteroids such as betamethasone in pharmaceuticals. For example, betamethasone can be combined with clotrimazole and optionally gentamicin. Betamethasone can be combined with salicylic acid, e.g. for use in the treatment of psoriatic skin conditions. The combination of betamethasone with calcipotriol is also a known therapy for psoriasis and hence the inclusion of calcipotriol in the compositions of the invention is envisaged.

Viewed from another aspect therefore, the invention provides a pharmaceutical composition, or kit as previously described further comprising clotrimazole, gentamicin salicylic acid, or calcipotriol or a salt, hydrate or solvate thereof.

In particular, we have found that the combination of a compound of formula (I), a corticosteroid partner and calciptriol, in particular the combination of betamethasone, calciptriol and a compound of formula (I), e.g. compound A defined herein, leads to an unexpected synergistic effect. We demonstrate in the examples, that this combination leads to a synergistic reduction in cell viabilty making compounds of formula (I) ideal adjuvants to other drugs in treatment of the inflammation and itching caused by a number of skin conditions such as psoriasis.

It is also envisaged that the compound of formula (I) as defined herein and corticosteroid partner such as betamethasone, could be combined with one or more secosteroid partners selected from the group consisting of calcipotriol or tacalcitol or a pharmaceutically acceptable salt, or a hydrate or solvate thereof. The calcipotriol is preferably in the form of its hydrate or its monohydrate.

The amounts of each compound present in the composition of the invention are determined in molar terms, and the ratio of each is preferably corticosteroid to compound (I) of 20:1 to 1:1 moles, such as 15:1 to 5:1 moles. There is therefore generally an excess of the corticosteroid in molar terms.

The amount of the compounds of the invention in the composition will often be determined by the physician depending on the dosage required.

### Skin Disorders

As noted above, the invention targets skin disorders, especially psoriasis and dermatitis. In particular, it is envisaged that the compositions of the invention may reduce inflammation and/or itchiness associated with the skin condition in question.

The combination therapy of the invention may have utility in treating a variety of different forms of dermatitis, such as atopic dermatitis or contact dermatitis. Thus, the compounds of the invention may be used to treat contact dermatitis such as allergic contact dermatitis or irritant contact dermatitis.

The nature of the allergan or irritant which causes the contact dermatitis can vary a lot and many people have different reactions to different allergans/irritants.

One of the most common causes of allergic contact dermatitis are plants of the Toxicodendron genus: poison ivy, poison oak, and poison sumac. Certain alkyl resorcinols such as bilobol found in Gingko biloba fruits are strong skin irritants. Other allergens include nickel, gold, balsam of Peru (Myroxylon pereirae), and chromium.

Common causes of irritant contact dermatitis are harsh (highly alkaline) soaps, detergents, and cleaning products. Irritant contact dermatitis can be divided into forms caused by chemical irritants and those caused by physical irritants. Common chemical irritants implicated include solvents (alcohol, xylene, turpentine, esters, acetone, ketones, and others); metalworking fluids (neat oils, water-based metalworking fluids with surfactants); latex; kerosene; ethylene oxide; surfactants in topical medications and cosmetics (sodium lauryl sulfate); alkalies (drain cleaners, strong soap with lye residues). Physical irritant contact dermatitis may most commonly be caused by low humidity from air conditioning. Also, many plants directly irritate the skin.

A further form of contact dermatitis is photocontact dermatitis. The skin condition is caused by exposure to ultraviolet light (320-400 nm UVA).

The invention may also lead to a treatment of atopic dermatitis. Atopic dermatitis is a type of eczema and is an inflammatory, chronically relapsing, non-contagious and itchy skin disorder.

Other less common forms of dermatitis to be treated include dermatitis herpetiformis, seborrheic dermatitis and stasis dermatitis.

By treating or treatment is meant at least one of:

(i). inhibiting the disease i.e. arresting, reducing or delaying the development of the disease or a relapse thereof or at least one clinical or subclinical symptom thereof, or

(ii). relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the pharmaceutical compositions of the invention are used therapeutically, i.e. to treat a condition which has manifested rather than prophylactically. It may be that the pharmaceutical composition of the invention is more effective when used therapeutically than prophylactically.

The pharmaceutical composition of the invention can be used on any animal subject, in particular a mammal and more particularly a human or an animal serving as a model for a disease (e.g., rat, mouse, pig, monkey, etc.). For example, in one use a pharmaceutical combination of the invention is used as a positive control in the animal subject to test other compounds for activity and/or side effects.

In order to treat a disease an effective amount of the active pharmaceutical composition needs to be administered to a patient. A “therapeutically effective amount” means the amount of a pharmaceutical composition that, when administered to an animal for treating a state, disorder or condition, is sufficient to effect such treatment. The “therapeutically effective amount” will vary depending on the pharmaceutical composition, the disease and its severity and the age, weight, physical condition and responsiveness of the subject to be treated and will be ultimately at the discretion of the attendant doctor.

It may be that to treat skin disorders according to the invention that the pharmaceutical composition of the invention has to be readministered at certain intervals. Suitable dosage regimes can be prescribed by a physician.

The pharmaceutical composition of the invention typically comprises the active components in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on. The pharmaceutical composition can also contain other active components, e.g. other drugs for the treatment of skin disorders.

It will be appreciated that pharmaceutical compositions for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The pharmaceutical compositions of the invention could also be formulated as nanoparticle formulations.

However, for the treatment of skin disorders, the pharmaceutical composition of the invention will preferably be administered topically. The pharmaceutical composition may therefore be provided in the form of a cream, gel, foam, salve or ointment.

The pharmaceutical composition of the invention may contain from 0.01 to 99% weight—per volume of the active material. The therapeutic doses will generally be between about 10 and 2000 mg/day and preferably between about 30 and 1500 mg/day of active components combined. Other ranges may be used, including, for example, 50-500 mg/day, 50-300 mg/day, 100-200 mg/day or active components combined.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The invention is described further below with reference to the following non-limiting examples and figures.

### DESCRIPTION OF FIGURES

FIG. 1 shows the results of the combination therapy of the invention. Co-treatment with cPLA2a inhibitor Compound A and corticosteroid Betamethasone 17, 21-dipropionate shows synergistic effects on decreasing keratinocyte cell proliferation and viability compared to each inhibitor alone. Average and standard deviation of 2-4 independent experiments performed in series of 8 technical replicates per treatment.

FIG. 2 shows co-treatment with corticosteroid betamethasone and vitamin D analogue calcipotriol shows synergistic effects on keratinocyte cell proliferation and viability compared to each inhibitor alone. Average and standard deviation of 2-4 independent experiments performed in series of 8 technical replicates per treatment. The use of betamethasone and calcipotriolis a known synergistic psoriasis treatment. FIG. 2 is added to show that the results of the present invention are comparable to the results in FIG. 2, proving the presence of synergy.

FIG. 3 shows a dose response of Compound A on immortalized keratinocyte cell line HaCat cell viability. Data presented are average and standard deviation of 3 independent experiments performed in series of 8 technical replicates per treatment. Star (*) represent significant difference compare to control (100%) (*P≤0.05; **P≤0.01; ***P≤0.001; ****P≤0,0001).

FIG. 4 shows a dose response of betamethasone on immortalized keratinocyte cell line HaCat cell viability. Data presented are average and standard deviation of 3 independent experiments performed in series of 8 technical replicates per treatment. Star (*) represent significant difference compare to control (100%) (*P≤0.05; **P≤0.01; ***P≤0.001; ****P≤0,0001).

FIG. 5 shows co-treatment with compound A and betamethasone has synergistic effects on human Keratinocyte cell viability compared to each inhibitor alone. Data presented are average and standard deviation of 3 independent experiments performed in series of 8 technical replicates per treatment. Star (*) represent significant difference in compare to control (100%) and in between inhibitors indicated with bars (*P≤0.05; **P≤0.01; ***P≤0.001; ****P≤0,0001).

FIG. 6 shows co-treatment of Compound A with Calcipotriol and Betamethasone has a synergistic effect on human Keratinocyte cell viability. Data presented are average and standard deviation of 1 independent experiments performed in series of 8 technical replicates per treatment. Star (*) represent significant difference in compare to control (100%) and in between inhibitors indicated with bars (*P≤0.05; **P≤0.01; ***P≤0.001; ****P≤0,0001).

### EXAMPLE 1

The following compounds were used in the Experiments:

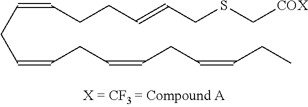

### Co-Treatment Compound A & Betamethasone:

### Methods:

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 2500 cells per well. Following 72 hours of cultivation, the cells were starved of serum in 0.25% FBS/DMEM overnight to halt proliferation, synchronize the cells and to increase cell sensitivity to treatment. On day 4, the cells were treated with cPLA2a inhibitor Compound A and corticosteroid Betamethasone 17, 21-dipropionate (Sigma Aldrich # B1152) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results:

Co-treatment with cPLA2a inhibitor Compound A and corticosteroid betamethasone shows synergistic effects on decreasing keratinocyte cell proliferation and viability compared to each inhibitor alone.

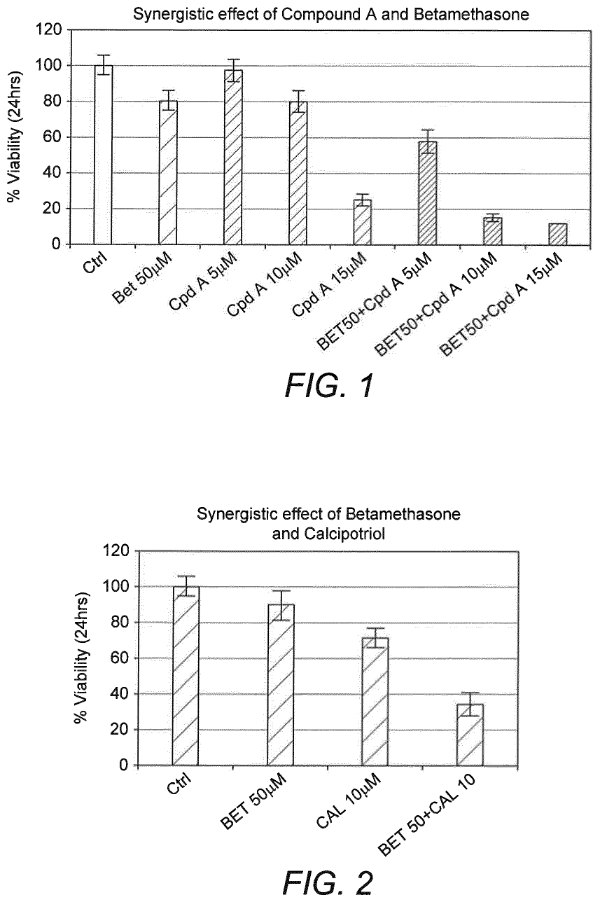

Initial experiments were performed to determine dose response of Compound A alone. The inhibitor slightly reduced cell proliferation and viability at 10 μM, whereas 5 μM did not show any affect (FIG. 1). On this basis, combination treatment experiments were designed in which sub-effective doses of the Compound A inhibitor and Betamethasone were combined.

Following 24 hours of treatment, 50 μM of Betamethasone and 5 or 10 μM Compound A alone showed little or no effect on reducing proliferation and viability of HaCaT cells, whereas 15 μM Compound A clearly reduced viability by ˜70%. However, when combining the sub-effective 5 and 10 μM doses of Compound A and Betamethasone, a significant ˜40% and ˜80% reduction of proliferation and viability was observed (FIG. 1). This observed trend of synergistic effects on cell proliferation and viability indicates beneficial effects of co-treatment of on skin disorders Several key pathways are dysregulated in skin disorders such as psoriasis and atopic dermatitis. cPLA2a inhibitors represent a promising adjuvant treatment to other drugs in treatment of the inflammation and itching caused by a number of skin conditions such as psoriasis and dermatitis.

### EXAMPLE 2

### Co-Treat Betamethasone & Calcipotriol

### Methods:

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 2500 cells per well. Following 72 hour of cultivation, the cells were starved of serum in 0.25% FBS/DMEM overnight to halt proliferation, synchronize the cells and to increase cell sensitivity to treatment. On day 4, the cells were treated with corticosteroid Betamethasone 17, 21-dipropionate (Sigma Aldrich # B1152) and vitamin Danalogue Calcipotriol hydrate (Sigma Aldrich # C4369) for 24 hours. On day 5, resazurin was added according to the manufacturer's instruction (RnD Systems, UK) and left to incubate for 2 hour incubator at 37° C. with 5% CO in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results:

Co-treatment with corticosteroid Betamethasone and vitamin Danalogue Calcipotriol shows synergistic effects on decreasing keratinocyte cell proliferation and viability compared to each inhibitor alone.

Betamethasone and Calcipotriol combination has already been established in treatment of Psoriasis. We here tested this established co-treatment to verify our methodology. Following 24 hours of treatment, 50 μM of Betamethasone and 10 μM Calcipotriol alone showed a 10% and 20% reduction of cell proliferation respectively which increased to a ˜35% reduction when given in combination (FIG. 2). This observed trend of synergistic effects on cell proliferation and viability show the relevance of the rezasurin assay and validate the previously reported beneficial effects of Betamethasone and Calcipotriol co-treatment on skin disorders.

### EXAMPLE 3

Compound A and betamethasone show dose response on immortalized keratinocyte cell line HaCat cell viability.

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:3-1:4 to ensure actively proliferating cells.

### Resazurin Assay:

Cells were seeded in 96 well plates in fully supplemented medium at a density of 3000 cells per well. Following 48-72 hour of cultivation, the cells were starved of serum in 0.25% FBS/DMEM overnight to halt proliferation, synchronize the cells and to increase cell sensitivity to treatment. Next day, the cells were treated with compound A and betamethasone dipropionate for 24 hours. Resazurin was added next day according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results

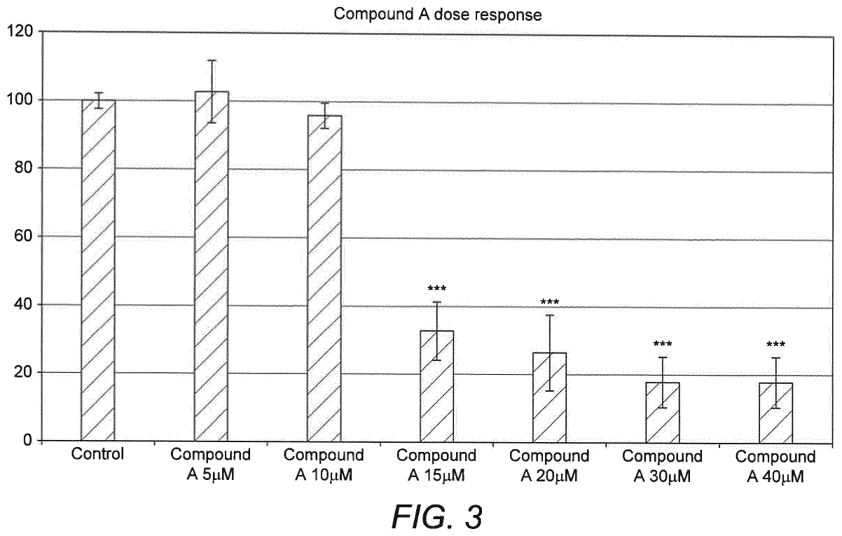

In this study, experiments were performed to determine dose response of Betamethasone dipropionate and compound A. Compound A was found to affect cell viability at 15 μM, whereas at doses 1-10 μM no signs of impairment in cell viability were observed (FIG. 3).

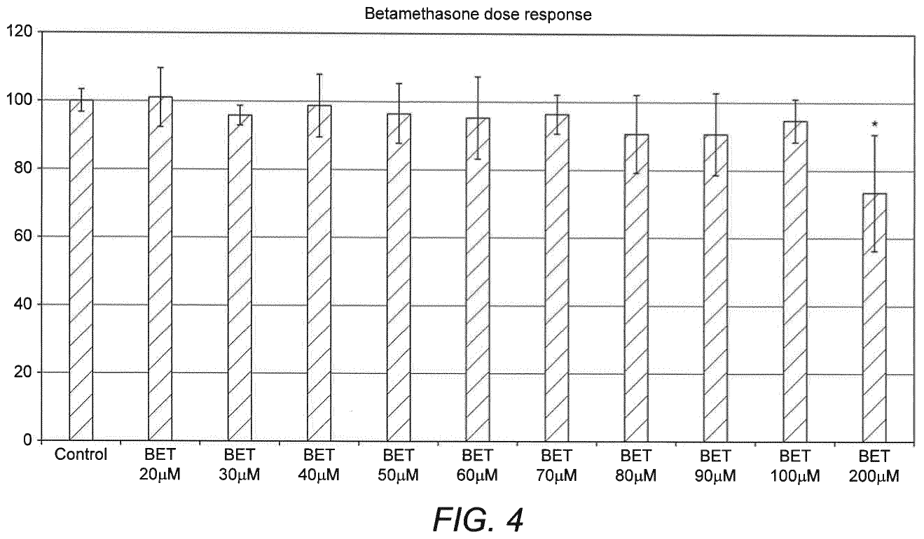

On the other hand, Hacat keratinocytes show resistance to betamethasone up to 200 μM (FIG. 4). The little effect seen is rather because of higher concentration of solvent DMS than Betamethasone (FIG. 4).

### EXAMPLE 4

Co-treatment with compound A and Betamethasone shows synergistic effects on immortalized keratinocyte cell line HaCat cell viability compared to each inhibitor alone. Example 4 employs the same assay as example 3.

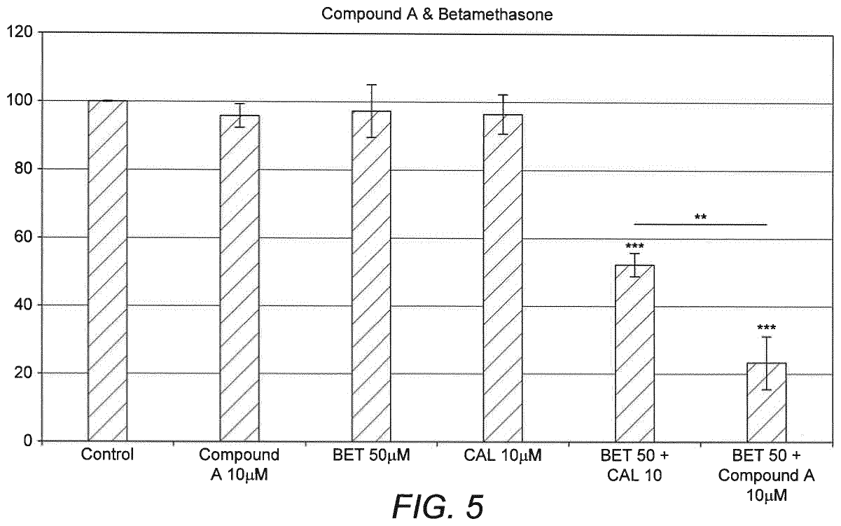

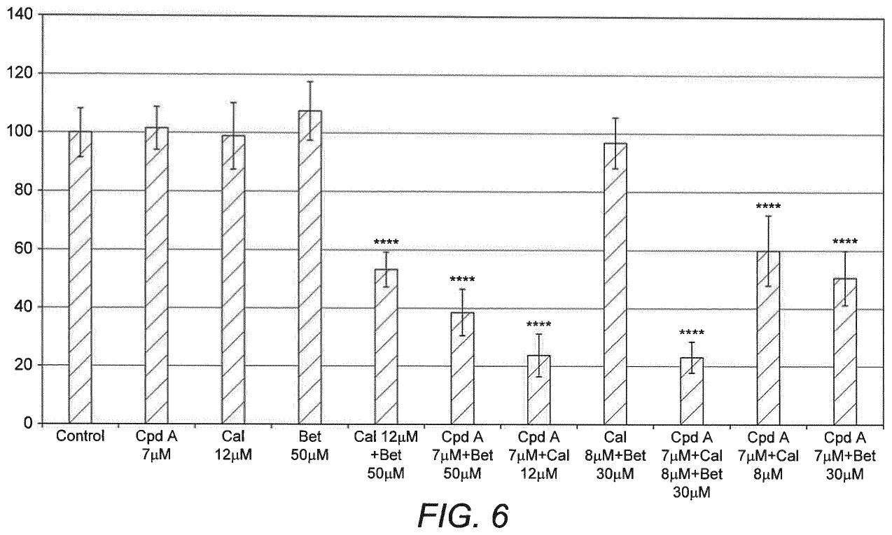

As noted in FIG. 3/4, the suboptimal dose of viability effect found in compound A and betamethasone was 10 μM and 50 μM (FIG. 3/4). Combination of the compound A and betamethasone were also compared with already established combination of betamethasone and calcipotriol. Following 24 hours of treatment, doses of betamethasone (50 μM) and calcitoriol (10 μM) shows 45% reduction in proliferation of HaCat cells. But the combination of compound A (10 μM) with Betamethasone (50 μM) modestly reduced an additional 25% more viability which is nearly 70% (FIG. 5). This observed trend of synergistic effects on cell viability indicate beneficial effects of co-treatment of compound A and betamethasone on skin disorders.

### EXAMPLE 5

Co-treatment of compound A with vitamin D analogue Calcipotriol and corticosteroid hormone receptor agonist Betamethasone shows synergistic effects on immortalized keratinocyte cell line HaCat viability both in dual and triple combination in compared to each inhibitor alone.

### Cell Culture:

The spontaneously immortalized, nontumorigenic skin keratinocyte cell line HaCaT was maintained in DMEM supplemented with 5% (v/v) FBS, 0.3 mg/ml glutamine and 0.1 mg/ml gentamicin at 37° C. with 5% CO₂ in a humidified atmosphere. Subculture using trypsin-EDTA was performed every 3-4 days with split ratio of 1:4 to ensure actively proliferating cells.

### Resazurin Assay:

Cells were seeded in 96 well plates in fully supplemented medium at a density of 3000 cells per well. Following 72 hour of cultivation, the cells were starved of serum in 0.25% FBS/DMEM overnight to halt proliferation, synchronize the cells and to increase cell sensitivity to treatment. Next day, the cells were treated with Compound A, vitamin D analogue Calcipotriol and corticosteroid hormone receptor agonist Betamethasone dipropionate for 24 hours. Resazurin was added next day according to the manufacturer's instruction (RnD Systems, UK) and left to incubate for 2 hour in incubator at 37° C. with 5% CO₂ in a humidified atmosphere before fluorescence was read at 544 nm excitation and 590 nm emission wavelength. The cells were observed under the microscope to evaluate possible morphology changes and signs of stress before addition of resazurin. The experiments were performed in series of 8 wells per treatment and repeated 2-3 times.

### Results:

Initial experiments were performed to determine dose response of Compound A and Calcipotriol and Betamethasone alone. Combination treatment was designed in which sub-optimal doses of the inhibitor Compound A and Calcipotriol and Betamethasone were combined. Combination of Compound A with Calcipotriol and Betamethasone were compared with already established combo of Betamethasone and Calcipotriol. Following 24 hours of treatment, 12 μM of Calcipotriol and 50 μM of Betamethasone shows 45% reduction of cell viability which increased to nearly 80% when same concentration of Calcipotriol is given with Compound A 7 μM. In addition, combination of Compound A 7 μM with 50 μM of Betamethasone cause 60% reduction.

In the same way, Calcipotriol 8M and Betamethasone 30 μM does not have any effect on cell viability. Nevertheless, addition of 7M to that dual combination cause almost 80% reduction, which is far better than the dual combination of same doses of Calcipotriol and Betamethasone with similar dose of Compound A.

These results show that Compound A may be used as adjuvant treatment to other drugs in treatment of the inflammation and itching caused by a number of skin conditions such as psoriasis.

1. A pharmaceutical composition comprising:
(A) at least one compound of formula (I):
R-L-CO—X (I)
wherein R is a C₁₀₋₂₄ unsaturated hydrocarbon group optionally interrupted by one or more heteroatoms or groups of heteroatoms selected from S, O, N, SO, SO₂, said hydrocarbon group comprising at least 4 non-conjugated double bonds;
L is a linking group forming a bridge of 1 to 5 atoms between the R group and the carbonyl CO wherein L comprises at least one heteroatom in the backbone of the linking group; and
X is an electron withdrawing group;
or a pharmaceutically acceptable salt, or a hydrate or solvate thereof; and
(B) one or more corticosteroid partners, preferably selected from the group consisting of betamethasone, clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a pharmaceutically acceptable salt, or a hydrate or solvate thereof, especially betamethasone or a pharmaceutically acceptable salt, or a hydrate or solvate thereof.

2. The pharmaceutical composition of claim 1 wherein the composition is a fixed combination or non-fixed combination.

3. A pharmaceutical composition as claimed in claim 1 for simultaneous, parallel, sequential or separate use comprising a kit comprising a first composition comprising at least one compound (I) and a pharmaceutically-acceptable diluent or carrier, and a second composition comprising at least one compound (B) and a pharmaceutically-acceptable diluent or carrier.

4. A composition as claimed in claim 1 wherein the compound (B) is betamethasone, dexamethasone or fluocortolone or a pharmaceutically acceptable salt, or a hydrate or solvate thereof.

5. A composition as claimed in claim 1 wherein the compound (B) is betamethasone or a pharmaceutically acceptable salt, or a hydrate or solvate thereof.

6. A composition as claimed in claim 1 wherein the compound (B) is betamethasone diproprionate, betamethasone valerate, betamethasone acetate or betamethasone sodium phosphate.

7. A composition as claimed in claim 1 wherein in formula (I), the group X is CHal₃, preferably CF₃.

8. A composition as claimed in claim 1 wherein in formula (I), the group R is a linear unsubstituted C₁₀₋₂₄ unsaturated alkylene group comprising at least 4 non-conjugated double bonds.

9. A composition as claimed in claim 1 wherein L is —SCH₂—.

10. A composition as claimed in claim 1 wherein said compound of formula (I) has the formula: 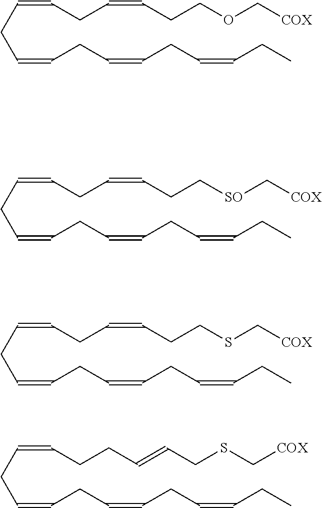
wherein X is as defined in claim 1, e.g. CF₃.

11. A composition as claimed in claim 1 wherein the compound of formula (I) is Compound A or Compound A2: 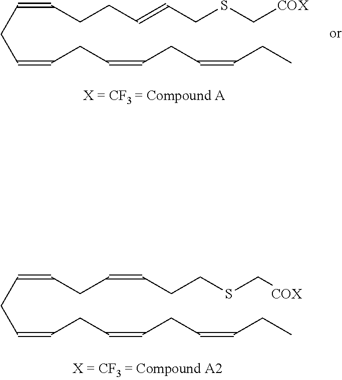
especially when compound (B) is betamethasone or a salt thereof.

12. A composition as claimed in claim 1 wherein the molar ratio of compound (A) to (B) in the composition is 1:1 to 1:20.

13. A composition as claimed in claim 1 further comprising clotrimazole, gentamicin, salicylic acid or calcipotriol or a pharmaceutically acceptable salt, or a hydrate or solvate thereof.

14. A composition as claimed in claim 13 further comprising calcipotriol or a pharmaceutically acceptable salt, or a hydrate or solvate thereof.

15. A composition as claimed in claim 14 comprising compound A, betamethasone or a pharmaceutically acceptable salt, or a hydrate or solvate thereof and calcipotriol or a pharmaceutically acceptable salt, or a hydrate or solvate thereof.

16. (canceled)

17. A method of treating, such as reducing symptoms of, or preventing a skin disorder such as psoriasis or dermatitis in a patient in need thereof comprising administering to said patient, preferably a human, an effective amount of a composition as claimed in claim 1.

18. A method of treating, such as reducing symptoms of, or preventing a skin disorder such as psoriasis or dermatitis in a patient in need thereof comprising administering to said patient, preferably a human, an effective amount of at least one compound of formula (I) and simultaneously, in parallel, separately or sequentially administering to said patient at least one compound (B), wherein formula (I) and compound (B) are defined in claim 1.

19. A method of treating such as, reducing symptoms of, or preventing a skin disorder such as psoriasis or dermatitis, in a patient in need thereof comprising:
(i) identifying a patient who has received either a compound of formula (I) as or a compound (B) respectively; and
(ii) administering to said patient an effective amount of either at least one compound (B) or at least one compound of formula (I) so that said patient is administered with both a compound of formula (I) and a compound (B), wherein formula (I) and compound (B) are as defined in claim 1.

20. A method of treating, such as reducing symptoms of, or preventing a skin disorder such as psoriasis or dermatitis in an animal subject in need thereof comprising administering to said animal an effective amount of a composition as claimed in claim 1.

21. A method of treating, such as reducing symptoms of, or preventing a skin disorder such as psoriasis or dermatitis in an animal subject in need thereof comprising administering to said animal an effective amount of at least one compound of formula (I) and simultaneously, in parallel, separately or sequentially administering to said animal at least one compound (B), wherein formula (I) and compound (B) are defined in claim 1.

22. The method of claim 20, wherein the animal subject is a rodent, monkey, or a pig.

23. The method of claim 21, wherein the pharmaceutical composition or the effective amount of compound of formula (I) and compound (B) is used as a positive control.

24. (canceled)

25. The pharmaceutical composition of claim 1 comprising betamethasone or a pharmaceutically acceptable salt, or a hydrate or solvate thereof optionally in combination with one or more additional corticosteroids or a pharmaceutically acceptable salt, or a hydrate or solvate thereof.

26. The pharmaceutical composition as claimed in 25, wherein the additional corticosteroid is selected from the group consisting of clobetasol, halometasone, dexamethasone, fluocortolone, desoximetasone, diflorasone, fluocinonide, flurandrenolide, halobetasol, amcinonide, halocinonide, triamcinolone, hydrocortisone, aclometasone, fluticasone, mometasone, clocortolone, fluocinolone, desonide, prednisone, prednisolone, and prednicarbate or a pharmaceutically acceptable salt, or a hydrate or solvate thereof.

27. A pharmaceutical composition as claimed in claim 1 in a form suitable for topical administration, e.g. a cream, gel, foam or ointment.
